(19) United States
(12) Patent Application Publication
(10) Pub. No.: US 2009/0005735 A1
(43) Pub. Date: Jan. 1, 2009

(54) Medical Device with Orientation Sensitive Priming Mechanism

(75) Inventors: Jakob Wikner, Taby (SE); Lennart Brunnberg, Tyreso (SE)

Correspondence Address: POTOMAC PATENT GROUP PLLC, P. O. BOX 270, FREDERICKSBURG, VA, 22404, US

(73) Assignee: SHL GROUP AB, Nacka Strand (SE)

(21) Appl. No.: 12/280,485

(22) PCT Filed: Feb. 19, 2007
(86) PCT No.: PCT/EP07/51561

§ 371 Date: Sep. 2, 2008

Related U.S. Application Data
(60) Provisional application No. 60/779,520, filed on Mar. 6, 2006.

(30) Foreign Application Priority Data
Mar. 3, 2006 (SE) 0600489-9

(51) Int. Cl. A61M 5/20

(52) U.S. Cl. 604/131

(57) ABSTRACT

The present invention relates to a device for a medical device, which medical device comprises a body (10, 12), a container (24) containing medicament, the container further comprising an expelling passage, and delivery drive means (36) for expelling said medicament, wherein said device comprises mechanical priming drive means (57) and mechanical priming activation (40) means being arranged and designed such that said mechanical priming activation means is capable of permitting activation of said mechanical priming drive means only when the medical device is substantially vertical with the expelling passage into an specific direction.

### TECHNICAL FIELD

The present invention relates to a medial device, and in particular a medical device having mechanical priming means, which ensures a safe and a correct priming of the medical device before the administering of a dose of medicament.

### BACKGROUND ART

The development of medial devices has become more and more directed towards the ability for the patient themselves to administer a medicament in an easy, safe and reliable way. Depending on the intended use and type of medicament, it has been developed a varying degree of automatic functions ranging from semi-automatic to fully automatic function, including e.g. injection devices penetration, injection, withdrawal and/or shielding of the needle. Other sub-functions have also been developed such as locking of a needle cover before and after use, means for varying the quantity of each dose, etc.

Generally, there are a type of medicaments that can be stored for a long time and that are packaged in containers or the like, containing a ready-to-use medicament in liquid state. There are also other type of medicaments that are a mixture of a medicament agent (e.g. lyophilized, powdered or concentrated liquid) and a diluent (e.g. water, dextrox solution or saline solution), wherein these type of medicaments can not be pre-mixed and stored for a long time because the medicament agent is unstable and can be degraded and loses its effect quickly. Hence, the patients have to perform the mixing within a limited time period prior to the delivery. In order to facilitate the mixing, containers or the like comprises at least two compartments, one compartment containing the medicament agent and the other compartment containing the diluent.

Conventionally injection devices are elongated devices with proximal and distal ends. Moreover, said injection devices comprise a container or the like, with one or more compartments containing a medicament to be delivered. Said conventional injection devices are further provided with a plunger rod that is adapted to be in contact with a piston provided inside the container or the like. When a patient or operator wants to administer a dose of medicament, a pointed needle is mounted on the distal end. Upon a force exerted on the piston by the plunger rod, the piston will move forward inside the container to expel the medicament from the container or the like. In many cases, the container contains some quantities of air because it is difficult to obtain a complete filling of medicament.

With a multi-compartment container the medicament agent has to be mixed with the diluent. A multi-compartment container or the like usually contains quite a lot of air, especially when the medicament agent is in powder form, which air is present when the medicament and the diluent are mixed. Further, also the needle contains air in its interior when it is mounted. It is also known that air can be sucked into the container if the patient lets the needle be mounted on the container or the like for a longer period of time and atmospheric conditions change.

This entrapped air is often desirable to get removed before injection. One danger is otherwise that the entrapped air is injected into the patient, which could cause injuries. Another aspect, even if the air would not cause direct harm, is that the first injected dose will be smaller than intended because of the amount of air, hence dose accuracy is reduced. There is further a psychological aspect. A patient may be worried if there is air entrapped and visual in a container of an injector, even though the air may not cause any harm. The mere knowledge that there is air in the container, and that the air might be injected, could be sufficient for a patient not to do want to use the device.

In order to remove the air or gas that is entrapped in the container or the like and/or the needle, priming needs to be performed by means of expelling a small dose of medicament. If at least a droplet or a short liquid jet is not expelled at the sharp point of the needle, another small dose of medicament should often be actuated. In view of that, sometimes the patients desire to set an unnecessary large dose in order to be sure that all air has been expelled.

Patent documents WO 9810814 A1 and U.S. Pat. No. 5,961,495 disclose a medical delivery device having a separate air shot button formed as a ring or a collar by which a predetermined amount of medicament can be expelled. A drawback with these devices is that the patients can prime these devices holding them at different positions, which do not ensure that the air or gas entrapped in the container or the like is expelled.

Other more advanced improvements have been made such as electronically controlled medical delivery devices, which comprise position sensors transmitting signals to a control unit indicating that e.g. priming/deaering cannot take place unless the longitudinal axis of the injection container is oriented in a predetermined direction. Such medical delivery devices are disclosed in US 2004/0186424 A1 and U.S. Pat. No. 6,869,413 B2. A drawback with these devices is that the electronics are dependent on batteries and are very sensitive to noise, moisture, water, etc.; which can result in malfunctions. Even more the manufacture of these devices is more expensive than the manufacture of mechanical devices.

Further, US2002/0177808 A1 discloses a safety device for preventing premature activation of an action e.g., administration and/or delivery of a drug into a living organism of a medicament delivery device. This safety device includes a housing that is arranged to enable such an action when the housing is in a desired orientation (e.g., horizontal, vertical). The housing includes a bowl having a concave cavity, a ball located in the cavity and a trigger having an actuator and a sleeve arranged to slide about the ball when the ball is in a predetermined position of the cavity to move the actuator and initiate the action. However, this document is completely silent about the priming of a container and specially a multiple chamber container in a vertical position. This document discloses that two vials that require reconstitution must be arranged vertical with their expelling passages directed downwards while the safety device is substantially horizontal and that the way of avoiding air after the reconstitution is through using a hydrophilic membrane. Thus the manufacture of this device is more expensive and complicated.

### DISCLOSURE OF INVENTION

There is thus a need to ensure that any entrapped air is expelled in a safe and reliable manner before medicament delivery.

According to a major aspect of the invention the present invention solves the above problems according to the features of claim 1.

Preferable embodiments and features of the invention are subject of the dependent claims.

According to a main aspect of the invention it is characterised by a device for a medical device, which medical device comprises a body, a container containing medicament, the container further comprising an expelling passage, and delivery drive means for expelling said medicament, wherein said device comprises mechanical priming drive means and mechanical priming activation means being arranged and designed such that said mechanical priming activation means is capable of permitting activation of said mechanical priming drive means only when the medical device is substantially vertical with the expelling passage into an specific direction.

For an injection device the expelling passage should be directed upwards and for an inhaler, in particular an inhaler having a pressurised metered dose canister, the expelling passage should be directed downwards.

The container could comprise at least one compartment containing a medicament and at least another compartment containing a diluent, a so called multi-compartment container. In that respect said mechanical priming drive means are capable of acting on said compartment for mixing the contents of said at least two compartments.

The device is preferably arranged such that said mechanical priming activation means is affected by gravitational force.

According to a further aspect of the invention, the device is characterised in that said mechanical priming activation means comprises at least one ball, that the mechanical priming drive means comprises components movable in relation to each other in order to activate the priming, and that said at least one ball is movable to a position allowing the components to interact only when said device is substantially vertical.

Preferably the at least one ball is made by a material of substantial density in order to promote distinct behaviour.

According to another aspect of the invention, the device is characterised in that said at least one ball, when said device is not held substantially vertical, blocks said interaction between said components.

According to yet an aspect of the invention, the device is characterised in that said ball, when said device is held substantially vertical, acts as an intermediate in the interaction between said components.

There are several benefits with the present invention. In contrast with known devices, the present device has not to rely on electronics, battery capacity and the like in order to have a reliable function of permitting priming only when the medicament delivery device is in the proper position. This ensures a safety for the user of the medicament delivery device. It is further less costly to manufacture such a safety feature in comparison to an electronic solution.

Preferably the gravitation is used to affect the mechanical priming activation means to permit or block the priming of the medicament delivery device. By using gravitation, a simple and yet reliable solution and function can be obtained with few components. It is thus not necessary to utilize spring means or other force components in order to obtain the desired mechanical function.

When using gravitation, it is an advantage to use a ball that can easily roll from one position to another depending on how the device is held. Either the position of the ball is used to block interaction between components that are used to activate the priming, or the ball is used as an intermediate component, for example transmitting a movement of one component to another for activation.

It is of course conceivable that the mechanical priming activation means could comprise pendulums, tilt members or the like.

These and other aspects and advantages of the present invention will become apparent from the following detailed description of the invention and from the accompanying drawings.

### BRIEF DESCRIPTION OF DRAWINGS

In the following detailed description of the invention, reference will be made to the accompanying drawings, of which

FIG. 1 shows a longitudinal cross-section of a first medicament delivery device comprising one embodiment of the present invention,

FIGS. 2a, b show an exploded view of the medicament delivery device according to FIG. 1,

FIG. 3 shows a detailed view of positioning means according to one embodiment of the present invention where the device may be primed,

FIG. 4 shows the view according to FIG. 3 when the device has been primed,

FIG. 5 shows the view according to FIG. 3 when the device is locked from being primed,

FIG. 6 shows a second medicament delivery device utilising the device according to the present invention,

FIG. 7 shows yet a third medicament delivery device utilising the device according to the present invention,

FIG. 8 shows a detailed view of a different embodiment according to the present invention, and

FIG. 9 shows a detailed view of yet another embodiment according to the present invention.

### DETAILED DESCRIPTION OF THE INVENTION

Typically, a medical device e.g. an inhaler, an injector, for delivering a pre-metered dose comprise a body, a container containing medicament, the container further comprising an expelling passage, and delivery drive means for expelling said medicament.

According to the main aspect of the invention said medial device comprises a device having mechanical priming drive means comprising a priming sleeve 40, a compression spring 48, an intermediate sleeve 52, a first set lateral ledges 62 attached to the rear end of a plunger rod (36, 83, 130) and ledges 60 of a setting sleeve 57, being arranged and designed to interact in relation to each other in order to activate the priming. Further, said device comprises mechanical priming activation means comprising at least one ball 56, being arranged and designed to interact with said mechanical priming drive means in order to block or permit the activation of said mechanical priming drive means.

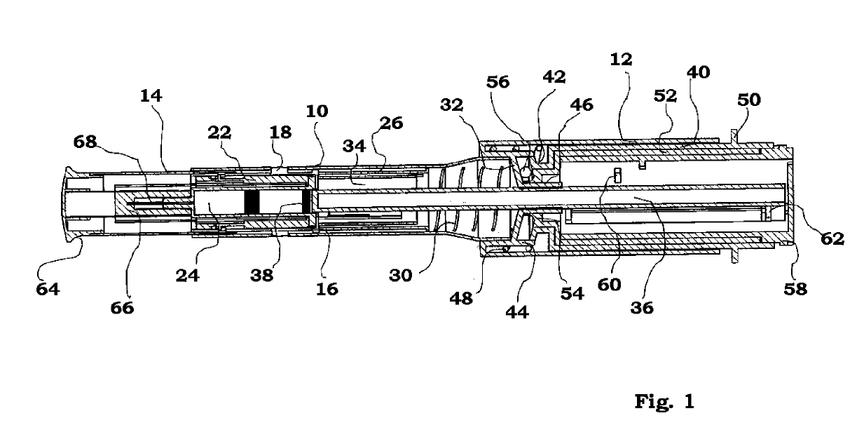

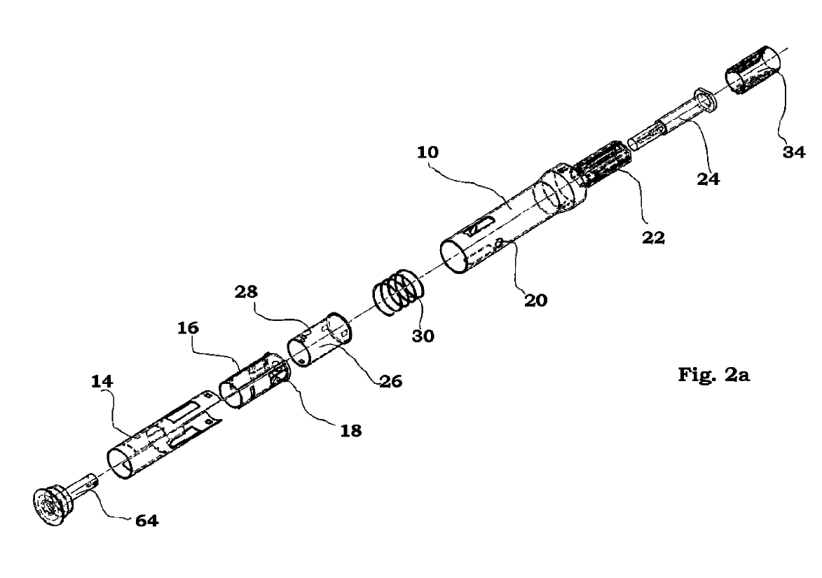

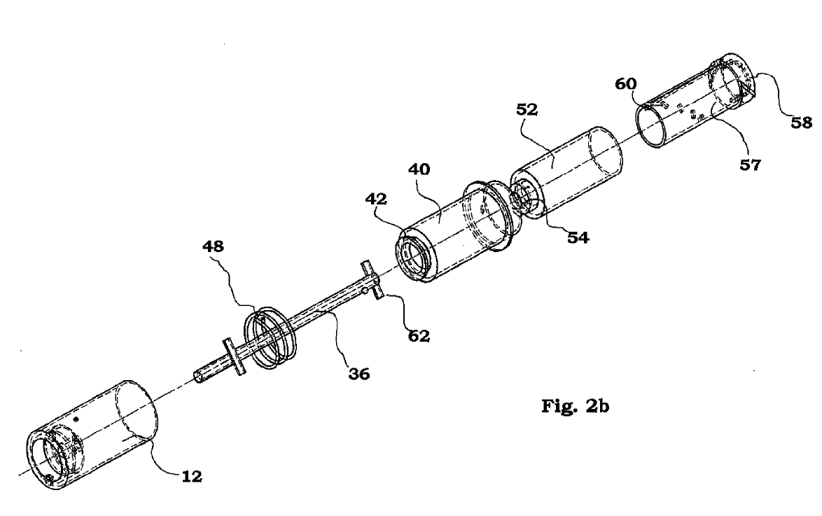

FIG. 1 shows a first medicament delivery device comprising the present invention. It comprises a body, a container containing medicament, the container further comprising an expelling passage, and delivery drive means for expelling said medicament. Said body comprises a front housing 10 and a rear housing 12 inter-connected to each other. Inside said body a container 24 containing medicament is arranged. In the embodiment shown, the container is arranged with one compartment containing medicament. It is however to be understood that there could be more than one compartments containing different contents to be mixed, e.g. one compartment containing medicament in powder form and another compartment containing some sort of diluent. Inside the front housing a needle shield 14 is slidably arranged. Inside the needle shield a medicament container sleeve 16 is attached to the front part via protrusions 18 fitting into holes 20 in the wall of the front housing and a container holder 22 holding the container 24 is arranged inside said container sleeve.

The delivery drive means of said first medicament delivery device being components comprising the needle shield 14, an activation sleeve 26, a compression spring 30, a control sleeve 34, a plunger rod 36 and a drive spring (not shown), being arranged and designed to interact in relation to each other in order to expel said medicament.

Said activation sleeve 26 is attached to the rear part of the needle shield 14 where the inner surface of the activation sleeve is arranged with a number of protrusions 28, the purpose of which will be explained below. The compression spring 30, hereafter named needle shield spring is arranged between a rear ledge of the activation sleeve and a wall 32 inside the rear housing. Inside the activation sleeve a control sleeve 34, hereafter named rotator, is arranged turnable around the longitudinal axis of the device. The outer surface of the rotator is arranged with grooves or ridges, arranged to co-act with the protrusions 28 of the activation sleeve 26, in a manner which will be explained below.

The plunger rod 36 extends through the device from a rear end of the container and in contact with a stopper 38 to a rear end of the rear housing.

The rear part of the device contains the priming sleeve 40 arranged inside and slidable in the longitudinal direction. The front part of the priming sleeve is arranged with a circumferential ledge 42, where the surface of the ledge slopes somewhat towards the centre of the sleeve. The diameter of the ledge substantially corresponds to the diameter of a cylindrical part 44 in the front part of the rear housing. In that area the wall 32 is arranged, having a central tubular passage 46 for the plunger rod. The compression spring 48 is arranged between an end wall of the rear housing and a ledge of the priming sleeve. In the embodiment shown the priming sleeve 40 protrudes somewhat outside the rear end of the rear housing. The protruding part of the priming sleeve is arranged with a circumferential ledge 50 so that the priming sleeve can be pushed towards the front of the device against the force of the compression spring.

In the rear end of the priming sleeve a knob 58 is arranged turnable and longitudinally movable with threads in relation to the priming sleeve 40. Further, the intermediate sleeve 52 has at its front end an annular ledge 54 with a diameter substantially corresponding to the inner diameter of the sloping ledge 42 of the priming sleeve 40. The inner edge of the annular ledge 54 is curved towards the front of the device. The ball 56 is placed in the space defined by the annular ledge 54 of the intermediate sleeve 52, the sloping ledge 42 of the priming sleeve, the cylindrical walls 44, 32 and the tubular part 46 of the rear housing 12.

Inside the intermediate sleeve 42, the setting sleeve 57 is turnable arranged. The rear end of the setting sleeve 57 protrudes outside the priming sleeve 40 and is designed as the knob 58. Further the inner surface of the setting sleeve is arranged with a number of ledges 60 intended to co-act with a first set lateral ledges 62 attached to the rear end of the plunger rod, which also will be explained in detail below.

The device is intended to function as follows. When the device is to be used a front protection cap 64 is removed, whereby a needle protection sheath 66 is removed from an expelling passage e.g. a needle 68. First the container has to be primed, i.e. to remove any air entrapped in the container. In order to ascertain that the air is removed before expelling a dose, the medical device has to be held such that the air bubbles are at the expelling point, i.e. at the needle. This means that the medical device should be held substantially vertical with the needle pointing upwards.

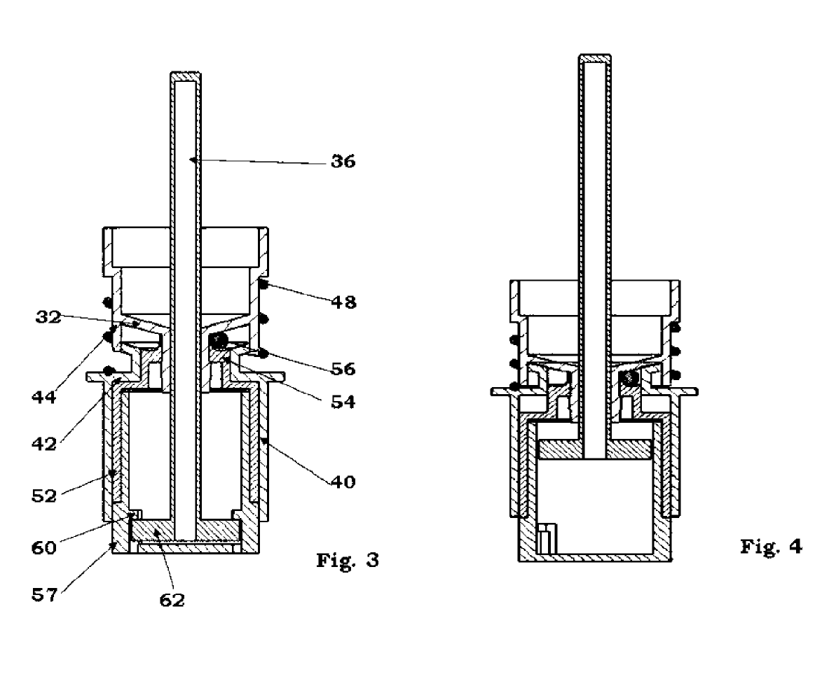

The present invention ensures that the priming can only take place when the medial device is in that position, due to the mechanical priming activation means. When the medical device is held substantially vertical the gravitation and the sloping surfaces of the priming sleeve 40 and of the intermediate sleeve 52 ensure that the ball 56 is paced in the position shown in FIG. 3. In this position it is possible to manually push the priming sleeve 40 towards the front of the device, FIG. 4, against the force of the compression spring 48. The movement of the priming sleeve causes the setting sleeve 57 to turn due to the interaction between the grooves and protrusions on the mating surfaces of the two sleeves. This in turn causes the ledge 60 that the lateral ledges 62 of the plunger rod 36 are resting on to be moved out of contact, thereby releasing the plunger rod. Due to the force of the plunger rod spring the plunger rod is moved forward and moves thus the stopper 38 in the container and expels the entrapped air through the needle. Because the device is in the substantially vertical position, it is assured that the air entrained in the container is removed before medicament delivery. The movement of the plunger rod is stopped when a second set of lateral ledges 62 come in contact with protrusions on the inner surface of the rotator 34. When the patient releases the priming sleeve 40, the compression spring 48 moves the priming sleeve back to its original position. The compression spring 48 further has the function of reducing the risk of jamming the ball when the priming sleeve is pushed towards the front of the device.

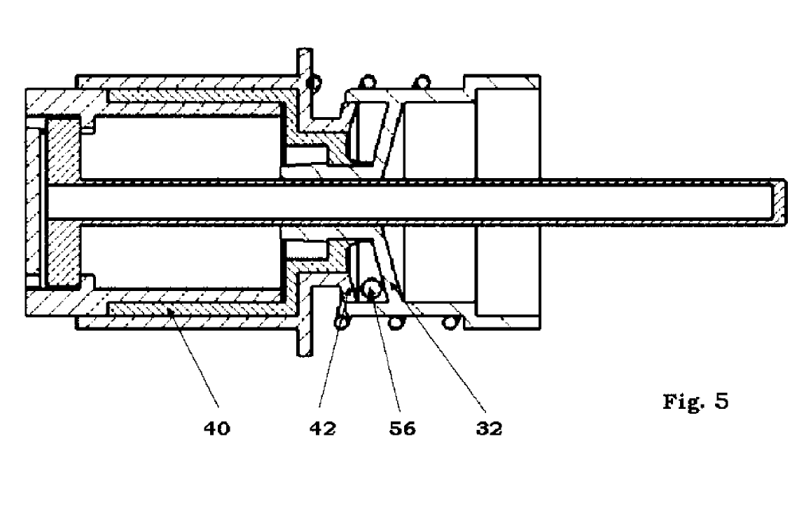

If however the device is not held in the substantially vertical position, the ball will roll towards the cylindrical wall into a position shown in FIG. 5. When the ball is in that position it is not possible to push the priming sleeve forward because the ball is an effective stop between the sloping surface 42 of the priming sleeve 40 and the inner wall 32 of the rear housing.

When the priming is completed the device is ready for medicament delivery. The patient first turns the dose setting knob 58 to a certain prescribed dose, which preferably is shown by indications on the dose knob. By turning the knob the setting sleeve is turned so that a certain protrusion on the inner surface of the dose setting knob is placed in line with the first set of literal ledges 62 of the plunger rod. It is thereby to be understood that the particular protrusions for particular dose amounts are placed at different longitudinal distances to allow longer or shorter travel of the plunger rod. The front end of the needle shield 14 is placed at the delivery site and the penetration of the needle 68 is done manually.

During penetration the needle shield is pushed inside the front housing. Because of the connection with the activation sleeve 26, this is also pushed towards the rear of the device. During the movement of the activation sleeve the interaction between the protrusions of the activation sleeve and the grooves or ridges of the rotator causes the rotator 34 to turn, whereby the second set of lateral ledges 62 of the plunger rod move out of contact with the protrusions on the inner surface of the rotator 34. The force of the plunger rod spring causes the plunger rod to move forward and thereby inject medicament into the patient. The movement of the plunger rod is again stopped when the first set of lateral ledges 62 abut the specific protrusions 60 for a certain dose placed on the inner surface of the setting sleeve.

When the medicament delivery is completed, the needle is withdrawn from the delivery site, whereby the needle shield 14 is pushed out again due to the needle shield spring. When in the extended position, protrusions on the outer surface of the needle shield fit into recesses in the inner surface of the front housing, which locks the needle shield in that position in order to avoid the risk of unintentional needle sticks.

Even though the mechanical priming activation means is shown to comprise a sleeve that is movable in the longitudinal direction, it could of course be arranged in other ways such as a knob, e.g. dose knob, that has a dual function setting the dose by turning and activating the priming by pushing.

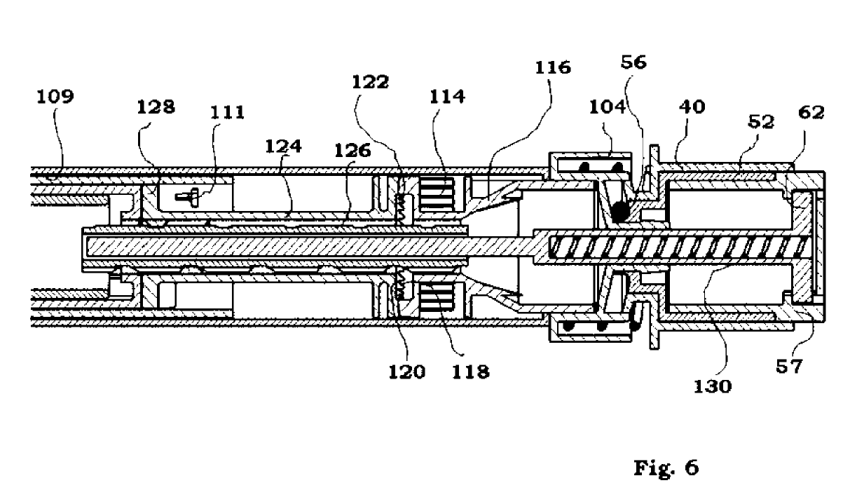

The present invention could of course be used with other types of medicament delivery devices. One type is shown in FIG. 6 which is a second medicament delivery device. Further, the delivery drive means of said second medicament delivery device being components comprising a needle shield 109, a flat spiral spring 114, a housing member 116, a first wheel 120, a plunger rod 126, a second wheel 128; being arranged and designed to interact in relation to each other in order to expel a medicament.

With this second medicament delivery device a dose delivery step is performed by rotating a dose wheel turning member 104 in clock-wise direction with predetermined equally large dose increment steps. When the turning member is rotated, the housing member 116 rotates as well, whereupon protrusions 118 on the end of the housing member slide over bevelled teeth 122 of the first wheel 120, i.e. when the turning member is rotated a protrusion 118 comes in engagement with the tooth that follows the tooth that said protrusion was previously in engagement with. Each time a protrusion 118 slides over a tooth 122, the dose is increased by one step and the increase of the dose with one step corresponds to a clock-wise rotation of the turning member with a predetermined number of degrees.

Each time the housing member is rotated clock-wise by one step, the flat spiral spring 114, winds up and accumulates energy that corresponds to the rotation of the dose wheel turning member 104 the number of degrees that corresponds to one clock-wise step turn.

The delivery device according to FIG. 6 is now ready to be set in a medicament delivery state. This is accomplished by pushing a needle shield 109 towards the distal end of the delivery device, preferably by pushing the proximal end of the needle shield against the patient's skin at the medicament delivery site. When the needle shield moves towards the distal end of the delivery device, stopper means 111 of the needle shield comes out of engagement with flanges 130 of the second wheel 128, which will not only set the second wheel 128 in a rotatable state but also the first wheel 120, since the wheels 120, 128 are connected via tubing 124.

The energy accumulated in the flat spiral spring 114 will now due to the output torque of the spring 114 when said spring now is free to unwind, rotate the housing member 116 counter clock-wise and also the wheels 120, 128 due to the acting of the protrusions 118 on the teeth 122.

The rotation of the wheels 120, 128 will urge a plunger rod 126 is further a predetermined distance towards the proximal end into a medicament container.

According to the present invention, the second medicament delivery device is arranged with a second rod 130, which is used for priming the medicament container before the above mentioned delivery steps. The second medicament delivery device is further arranged with a priming device as described above. Thus, when the device is to be primed, it has to be held substantially vertical as described above. In this position it is possible to manually push the priming sleeve 40 towards the front of the device. The movement of the priming sleeve causes the setting sleeve 57 to turn due to the interaction between the mating surfaces of the two sleeves. This in turn causes the ledge 60 of the setting sleeve 57, that the lateral ledges 62 of the plunger rod 130 are resting on to be moved out of contact, thereby releasing the second rod to be moved forward and perform the priming step.

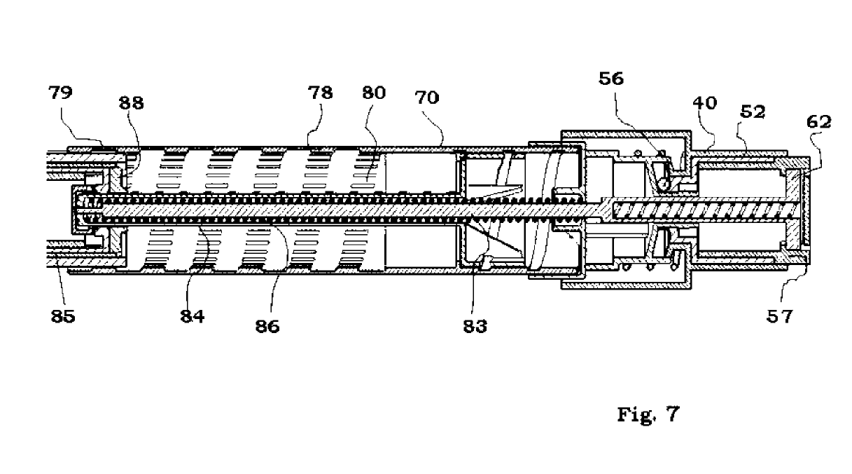

FIG. 7 shows yet an embodiment of a third medicament delivery device in which the present invention may be utilised. Further, the delivery drive means of said third medicament delivery device being components comprising a needle shield 85, a plunger rod 84, a drive spring 86 and a wheel 88; being arranged and designed to interact in relation to each other in order to expel a medicament. With this third medicament delivery device a dose is set in a first dose delivery step by rotating a back cover 70 clock-wise. When rotating the back-cover, the entire back cover will through rotation move towards the proximal end of the device as thread 78 on the inner surface of the back cover is in engagement with thread 79 on the outer surface of a proximal part of the device. As the back cover 70 move towards the proximal end of the device, recesses 80 of the thread 78 slide over corresponding protrusions (not shown) on the exterior of the proximal part. Each time a recess 80 slides over such a corresponding protrusion, the dose is increased by one step.

As the back cover moves in steps towards the front end, also the plunger rod spring 86 in the interior of the plunger rod 84 is compressed and step-wise accumulates a spring force corresponding to the predetermined distance that the back-cover 70 moves towards the front end. The higher dose set, the greater spring force accumulated in the spring 86.

The third medicament delivery device is now ready to be set in a medicament delivery state. This is accomplished by pushing the needle shield 85 towards the distal end of the delivery device. When the needle shield moves towards the distal end of the delivery device, a stopper means of the needle shield come out of engagement with teeth of a wheel 88. Due to the accumulated spring force in the plunger rod spring 86, the plunger rod will now, be screwed into a container housing and moves thus towards the front end.

During dose delivery, when the plunger rod 84 is forced into the container housing, the wheel 88 is rotated along with the rotating plunger rod and travels along its longitudinal axis. When the dose has been delivered the user releases the needle shield, by for instance simply removing the device from the delivery site.

As with the previous embodiments, also this is arranged with a mechanical priming device functioning in very much the same manner as the previously mentioned medical delivery device. Thus, before the mentioned delivery step, the priming has to take place, which according to the present invention has to be performed substantially vertical. As with the previous embodiments, when the device is held vertically, the ball is placed in the position shown, whereby it is possible to manually push the priming sleeve 40 towards the front of the device. The movement of the priming sleeve causes the setting sleeve 57 to turn due to the interaction between the grooves and protrusions on the mating surfaces of the two sleeves. This in turn causes the ledge 60 that the lateral ledges 62 of a second plunger rod 83 are resting on to be moved out of contact, thereby releasing the second plunger rod 83.

Within the meaning of substantially vertical, it is to bee understood a position where it is ensured that the entrapped air is at the expelling point, i.e. at the inner end of the needle. This means that the medical device should be held within a conical angle of between about 0° to 150°. The maximum angle is determined by the angle of the sloping surface of the priming sleeve, the weight of the ball and the friction between the ball and the surface.

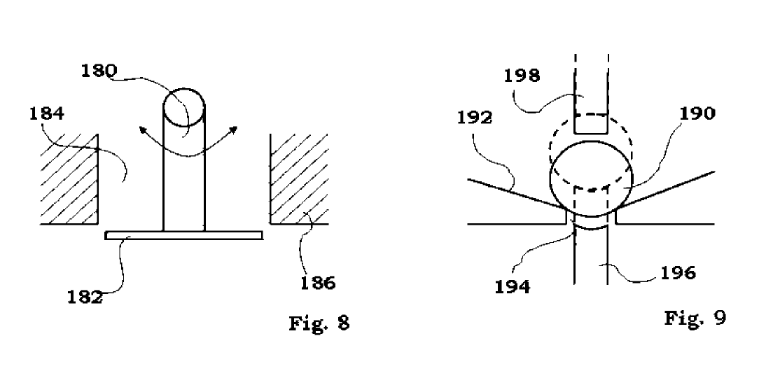

It is of course conceivable to other types and solutions of mechanical priming activation means for ensuring the proper position of the device when priming. FIG. 8 shows a variant where a sort of pendulum 180 is used, which for example could be connected in a suitable way to the priming sleeve. When the device is held vertically the lateral ledges 182 can pass through a passage 184 and thereby activate a priming, but when the device is held inclined the ledges will come in contact with the edges 186 of the passage and can thus not pass through the passage.

Another example is shown in FIG. 9 also using a ball 190 and sloping surfaces 192. When the device is held in the proper position, the ball is placed over a central passage 194. When a pin 196, for example attached to the priming sleeve, under the ball is moved upwards, it comes in contact with the ball, which also is moved upwards and comes in contact with an activation pin 198 for priming. However if the device is held inclined, the ball is moved from the central passage. If the pin then is moved upwards, it does not come in contact with the activation pin.

With a proper design and dimensions of the components according to the present invention, it is possible to control and adjust at which angle deviation from vertical that it should no longer be possible to prime the device.

It is to be understood that the embodiments described above and shown in the drawings are to be regarded as only non-limiting examples of the invention and that it can be modified within the scope of the patent claims. For example the device according to the present invention may also be used in connection with inhalers, such as nebulisers and the like having medicament compartments including air, which compartments need to be primed before use. The present invention could also be used for pressurised metered dose inhalers where it is important that the device is held substantially vertical with the canister opening facing downwards when priming the canister. The present invention is then used to prevent priming of the canister when held in an improper position, which ensures that the canister is primed in the proper fashion.

1.-13. (canceled)

14. A medicament delivery device, comprising:
a front housing;
a rear housing;
a container containing medicament arranged within the front housing and having an expelling passage;
delivery drive means for expelling the medicament;
first mechanical priming drive means connected to the rear housing;
second mechanical priming drive means connected to the delivery drive means and movable in relation to the first mechanical priming drive means and the rear housing;
mechanical priming activation means movably arranged in relation to the first mechanical priming means respective the second mechanical priming drive means and the rear housing in such a way that under a predetermined specific direction of the expelling passage the mechanical priming activation means permits the second mechanical priming drive means to be moved; and
spring means arranged to interact with the rear housing and the second mechanical priming drive means in such a way that the spring means exerts a force on the second mechanical priming drive means for holding the second mechanical priming drive means apart from the mechanical priming activation means, thereby avoiding jamming of the mechanical priming activation means.

15. The device of claim 14, wherein the mechanical priming activation means comprises at least one ball, or pendulums, or tilt members.

16. The device of claim 15, wherein the at least one ball is movable to a position enabling the second mechanical priming drive means to be moved when the device is substantially vertical.

17. The device of claim 16, wherein the at least one ball, when the device is held substantially vertical, acts as an intermediate in interaction between the first mechanical priming drive means respective second mechanical priming drive means and the rear housing.

18. The device of claim 16, wherein the at least one ball, when the device is not held substantially vertical, blocks movement of the second mechanical priming drive means.

19. The device of claim 18, wherein the at least one ball, when the device is held substantially vertical, acts as an intermediate in interaction between the first mechanical priming drive means respective second mechanical priming drive means and the rear housing.

20. The device of claim 14, wherein the device is provided in an injection device and the expelling passage is directed upwards.

21. The device of claim 20, wherein the mechanical priming activation means comprises at least one ball, or pendulums, or tilt members.

22. The device of claim 21, wherein the at least one ball is movable to a position enabling the second mechanical priming drive means to be moved when the device is substantially vertical.

23. The device of claim 22, wherein the at least one ball, when the device is held substantially vertical, acts as an intermediate in interaction between the first mechanical priming drive means respective second mechanical priming drive means and the rear housing.

24. The device of claim 22, wherein the at least one ball, when the device is not held substantially vertical, blocks movement of the second mechanical priming drive means.

25. The device of claim 24, wherein the at least one ball, when the device is held substantially vertical, acts as an intermediate in interaction between the first mechanical priming drive means respective second mechanical priming drive means and the rear housing.

26. The device of claim 14, wherein the device is provided in an inhalation device having a canister of a pressurized metered dose inhaler, and the expelling passage is directed downwards.

27. The device of claim 26, wherein the mechanical priming activation means comprises at least one ball, or pendulums, or tilt members.

28. The device of claim 27, wherein the at least one ball is movable to a position enabling the second mechanical priming drive means to be moved when the device is substantially vertical.

29. The device of claim 28, wherein the at least one ball, when the device is held substantially vertical, acts as an intermediate in interaction between the first mechanical priming drive means respective second mechanical priming drive means and the rear housing.

30. The device of claim 28, wherein the at least one ball, when the device is not held substantially vertical, blocks movement of the second mechanical priming drive means.

31. The device of claim 30, wherein the at least one ball, when the device is held substantially vertical, acts as an intermediate in interaction between the first mechanical priming drive means respective second mechanical priming drive means and the rear housing.

32. The device of claim 14, wherein the second mechanical priming drive means includes a priming sleeve and a setting sleeve, and the first mechanical priming drive means includes an intermediate sleeve.

33. The device of claim 32, wherein the priming sleeve comprises sloping surfaces on which the ball can roll, for controlling the movement of the ball between an activation position and a non-activation position.
